(19) United States
(12) Patent Application Publication
(10) Pub. No.: US 2017/0057721 A1
(43) Pub. Date: Mar. 2, 2017

(54) SPLITTABLE OUTER PACKAGING FOR ABSORBENT PERSONAL CARE ARTICLES

(71) Applicant: KIMBERLY-CLARK WORLDWIDE, INC., NEENAH, WI (US)

(72) Inventors: HyeJin Lee, Seoul-si (KR); SeongDae Roh, Yongin-si (KR); JongSoo Lee, Yongin-Si (KR); Alice Susan Gordon, Roswell, GA (US)

(21) Appl. No.: 15/251,972

(22) Filed: Aug. 30, 2016

Related U.S. Application Data
(60) Provisional application No. 62/212,046, filed on Aug. 31, 2015.

(51) Int. Cl. B65D 75/52; A61F 13/551; B65D 85/62; B65D 75/58

(52) U.S. Cl. CPC B65D 75/527; B65D 75/5827; A61F 13/5514; B65D 85/62

(57) ABSTRACT

A relatively large splittable outer packaging includes a splitting structure along at least one side face panel, for separating two portions of the outer packaging into at least one relatively shorter outer package. Upon separation of the two portions, individually wrapped absorbent articles having sealed edges on the wrappers and that are contained in the outer packaging, are exposed at openings in the at least one relatively shorter outer package, for ease of dispensing.

### PRIORITY

This application is a continuation of application Ser. No. 62/212,046, entitled “SPLITTABLE OUTER PACKAGING FOR ABSORBENT PERSONAL CARE ARTICLES” and filed in the U.S. Patent and Trademark Office on Aug. 31, 2015. The entirety of the prior application is hereby incorporated by reference in this application.

### FIELD OF THE INVENTION

The present invention is generally directed to packaging for absorbent personal care articles. In particular, the present invention is directed to outer packaging for absorbent personal care articles, which may be transfigured into multiple packaging formats to suit a consumer's particular article-storage needs.

### BACKGROUND OF THE INVENTION

Absorbent personal care articles, such as feminine-care pads, napkins, and panty liners, and adult-care absorbent inserts (for undergarments) and absorbent pants, are frequently offered for sale as multiple individually-wrapped articles (each an inner package) contained within a single outer enclosure, so as to help preserve the cleanliness of such articles prior to use. For example, at least one compressed stack or row of individually-wrapped articles (i.e. each article in its own individual pouch-wrapper) are typically packed within either an outer-box, carton, or flexible-material container (hereinafter all referred to as “outer packaging”), such as a film or foil-based bag. The wrappers themselves are typically made from a film or nonwoven sheet.

Consumers typically favor storing such outer packaging in a conveniently located pantry or residential cabinet prior to use. Smaller outer packaging (which contains a single stack of articles) is capable of being stored in bathroom or desk drawers which frequently have a relatively short height. Such small outer packaging also provide consumers with discretion during their workday, as such packaging may also be stored discretely in private spaces within a consumer's personal work environment. In contrast, larger outer packaging (i.e. a bulk volume purchase), which typically contains several parallel stacks of individually-wrapped absorbent articles, offer the advantage of lower purchase price per absorbent article. Unfortunately however, such larger outer packaging is often too big to conveniently fit in easy-to-access desk or bathroom drawers (as the dimensions of such packaging exceed the height dimension of the drawers). As a result, such larger outer packaging must be stored in less convenient, out-of-the-way locations such as bathroom under-cabinets, or closets. There is therefore a need for an outer packaging which accommodates large numbers of individually-wrapped absorbent articles, and which is also adaptable, so as to be capable of being stored in convenient residential or discrete, work-related locations.

As a result of stacked absorbent articles being contained under pressure in an outer packaging, consumers have sometimes had difficulty in removing a single individual absorbent article at a time, without inadvertently also pulling multiple articles from the package. This is particularly problematic with larger outer packaging, which often has one article-retrieval opening. It is not uncommon for multiple articles to spill out from the one opening as a single article is removed. There is therefore a need for an outer packaging configuration which helps facilitate the removal of a single absorbent article from a compressed stack of articles.

As noted previously, consumers have typically stored larger outer packaging in a single, out-of-the-way location. As a result, a common practice has been for consumers to periodically remove a grouping of loose, absorbent articles from the large outer packaging, and place the loose articles in more proximate, secondary storage containers within their residence, work environment, or on their person, for easier accessibility later. For example, consumers frequently take a small handful of individual absorbent articles from a large outer packaging and place them in either their purse (in the case of feminine care panty liners) or other close cabinet/drawer locations for later use. In this fashion, consumers avoid having to return to the large outer packaging storage location, each time a single absorbent article is needed for use. However, such loose absorbent articles frequently shift about within the more proximately located drawers, purses, or other secondary storage containers, as the containers are repeatedly moved, opened, and closed. As a result of such shifting, the loose absorbent articles become disorganized, and may also become soiled (at least on their exterior surfaces) from coming in contact with cosmetics, toiletries, or other objects which are also contained in the secondary storage container. The general disarray of a consumer's drawers or purses may also lead to the crumpling or damage of the absorbent articles. There is therefore a need for a large outer packaging which also allows a consumer to store reduced numbers of absorbent articles in multiple locations in an organized fashion, while still providing protection from incidental soiling (pre-use soiling), and while benefitting from the high volume, discounted individual article, purchase-price.

As noted previously, small outer packaging often includes a single stack of absorbent articles. Such small outer packaging may include a slit, slot-like openings, or other dispensing mechanisms, which assist the consumer in dispensing individual articles one-at-a-time, from the outer package. In such formats, the slits or openings are typically narrower in width than the width of the absorbent article to be withdrawn through them, making article withdrawal cumbersome. Furthermore, the placement of such slits is awkward, and potentially incompatible with most larger-volume packaging configurations, since the compression of a large volume of articles in a package is often greater than in smaller packages, and the larger packages are frequently formed from polymer films. Such film structure makes a slit feature difficult to accommodate, without use of more expensive, bag-reinforcement materials at least at the slit location. There is therefore a need for a large outer packaging which offers the benefit of discounted consumer pricing as a result of greater article count, but which package also includes easy-dispensing mechanisms. There is a further need for a large outer packaging that can be transfigured into more manageable, shorter-sized outer packages depending on a consumer's particular article storage desires. There is still a further need for such large outer packaging which can be transfigured into multiple shorter, asymmetric shaped packages (i.e. nonequal portions), to accommodate either different types/sizes of absorbent articles and/or differently sized/shaped secondary storage containers.

In order to provide consumers with the ability to split relatively larger volume storage packaging for absorbent articles, into more consumer-friendly sized packaging, package designs have been developed which include perforation or separation mechanisms to allow consumers to break apart a single, relatively larger storage package into multiple, relatively smaller packages. Such package configurations are exemplified for instance, by U.S. Pat. Nos. 2,967,610 to R. W. Ebert et al., 3,090,483 to A. F. Altree et al., and 5,361,905 to McQueeny et al. As seen for example in FIG. 4C of McQueeny, individual unwrapped absorbent articles are present in multiple, side-by-side compressed stacks. Once the relatively larger, single packaging is separated along perforation lines, one stack of unwrapped individual articles is retained within each of two relatively shorter, but equally sized packages. While offering the convenience of dispensing from relatively smaller packages, each smaller package configuration then requires folded individual absorbent articles to be pulled (through an opening formed in each of the separated packages) at the article folded edges themselves. To accomplish this, consumers need to insert their thumb and forefinger about the side edges of the packed individual absorbent article (or multiple articles) and pull the article(s) out of the relatively smaller packaging. The consumer is required to essentially insert their fingers between adjacent articles in a stack (touching the side edges of multiple articles themselves), in order to pull an article from the stack. Such action likely again leads to the withdrawal of multiple articles at any given time, if such stacks are under high compression. Further, such dispensing allows for the possible soiling of one or more of the absorbent articles themselves upon removal, especially if the consumer's hands are themselves already soiled. Such soiled hands are not uncommon when a consumer or caregiver is in the process of replacing a “used” absorbent article with a “fresh” absorbent article. Also, if the consumer desires for convenience, to place these loose unused articles in a secondary container for use at a later time as previously discussed, further article contamination is possible. There is therefore a need for a relatively larger outer packaging that is capable of transfiguring into relatively smaller outer packages, while facilitating the easy removal of individually-wrapped absorbent articles contained therein, and protecting such articles (and adjacent articles) from possible pre-use soiling.

In order to facilitate the removal of a single absorbent article from a package in which multiple unwrapped articles are present in a compressed stack, gripping devices have been developed for attachment to individual, unwrapped absorbent articles. For example, as seen in United States Patent Publication 200710175789A1 to Ronnberg et al., a separate gripping device may be attached to an unwrapped absorbent article (or partially unwrapped absorbent article) that is contained in a stack of articles. However, should a consumer wish to store such individual unwrapped articles in a secondary storage container, there is still a possibility for incidental soiling to occur prior to article use. Further, the use of such gripping devices requires additional article manufacturing materials (at added cost), specific article orientation within a package, and complex manufacturing processes. Another approach for facilitating the removal of compressed absorbent articles is described in U.S. Pat. No. 7,168,563 to Zander et al. While such reference describes the use of an open sheath to be placed over a folded absorbent article to assist in its removal from a stack of articles under compression, as well as the use of open sheaths with sealed edges, such reference describes the use of a single opening for article dispensing. There is therefore a need for a relatively larger outer packaging that is capable of transfiguring into multiple, relatively smaller outer packages, while facilitating easy removal of the individually-wrapped absorbent articles contained therein (without added complexity in manufacturing, and additional material), and also, while completely avoiding exposure of the articles themselves to the outside environment so as to maintain article cleanliness prior to use.

### SUMMARY OF THE INVENTION

A splittable outer packaging has a length direction, a height direction, and a thickness direction, and further has at least one side face panel and an upper and lower portion. The splittable outer packaging includes a splitting structure for separating the splittable outer packaging upper and lower portions from one another, said structure included along said at least one side face panel. At least one stack of individually-wrapped absorbent articles with each absorbent article being completely wrapped in a wrapper, is included within at least one of the upper and lower portions, and alternatively each of the upper and lower portions. Each wrapper has at least one sealed edge. At least a portion of the individually-wrapped absorbent articles in the at least one stack, have their sealed edges oriented within the splittable outer packaging such as to be parallel with the splittable outer packaging thickness direction and generally adjacent to the splitting structure. In one embodiment, the sealed edges are all aligned with the splitting structure.

In an alternative of the splittable outer packaging, the splitting structure is selected from the group consisting of one or more lines of weakness, one or more perforation lines, or separated seams held together with a fastening device, such as a tape, ribbon, cord, band, or outer wrapper. In still a further alternative embodiment, the splitting structure is a fitted or screw-on edge. In yet a further alternative embodiment, the splittable outer packaging includes an upper portion having at least one stack of individually-wrapped absorbent articles, and a lower portion having at least one stack of individually-wrapped absorbent articles. In yet a further alternative embodiment, the upper portion includes one type of individually-wrapped absorbent articles, and the lower portion includes a different type of individually-wrapped absorbent articles. In still another alternative embodiment, the upper portion includes one stack of individually-wrapped absorbent articles, and the lower portion includes multiple stacks of individually-wrapped absorbent articles. In another alternative embodiment, the lower portion includes at least two stacks of individually-wrapped absorbent articles.

In yet a further alternative embodiment, the upper and lower portions each contain stacks with different numbers of individually-wrapped absorbent articles in each stack. In still another alternative embodiment, the stacks of individually-wrapped absorbent articles of the upper and lower portions each include visually differentiated wrappers.

In another alternative embodiment, the splittable outer packaging includes a different outer packaging appearance for each portion. Such outer packaging appearance may differ between upper and lower portions for example by differences in color, gloss level, packaging finish, or by the inclusion or placement of translucent or clear packaging materials.

In another alternative embodiment of the invention, the splittable outer packaging may include a frontside, backside, and two additional side face panels, in addition to a top and bottom face panel. In such an embodiment, the splitting structure may be located on either three of the front, back, and two side face panels or four of the front, back, and two side face panels.

In yet another alternative embodiment of the invention, the splittable outer packaging upper and lower portions, each include at least one stack of individually-wrapped absorbent articles. A plurality of wrapper sealed edges of individually-wrapped absorbent articles from at least one stack of each portion, are oriented such that they are parallel with the thickness direction of the splittable outer packaging and generally adjacent to the splitting structure. In one embodiment, they are aligned with the splitting structure.

In still a further alternative embodiment, the individually-wrapped absorbent articles are folded absorbent absorbent articles selected from the group consisting of feminine hygiene absorbent personal care articles and adult incontinence absorbent personal care articles. In another alternative embodiment, the individually-wrapped absorbent articles are selected from the group consisting of panty liners, sanitary pads, incontinence inserts, incontinence pads, and incontinence pants. In another alternative embodiment, the wrappers include a wrapper body portion, and the wrapper sealed edges extend outwardly away from the wrapper body portion. In another alternative embodiment, the wrappers include two opposing wrapper sealed edges. In still another embodiment, the wrappers include four wrapper sealed edges. In yet another alternative embodiment, the wrappers are visually distinguishable so that adjacent wrappers of individually-wrapped absorbent articles are visually different or distinguishable from one another.

In another alternative embodiment, the upper and lower portions each include at least one stack of individually-wrapped absorbent articles, and the stacks are generally parallel and adjacent one another. Wrapper sealed edges from each stack at least partially interlap with wrapper sealed edges of an adjacent stack, while in the splittable outer packaging. In still a further alternative embodiment, rather than interlapping, wrapper sealed edges from adjacent stacks of upper and lower portions are in physical contact, within the splittable outer packaging.

In still another alternative embodiment of the invention, the splittable outer packaging further includes visual cues on the outside of the splittable outer packaging, to highlight either the presence of, or the functionality of the splitting structure within the splittable outer packaging. In another alternative embodiment, the upper and lower portions each include at least one stack of individually-wrapped absorbent articles, and at least the wrapper sealed edges from each stack are colored differently between each portion.

In yet another alternative embodiment, the splitting structure is present on the at least one side face panel at a certain height (splitting structure height), and the height of each wrapper within at least the lower portion is larger than the splitting structure height. In still a further alternative embodiment, the splitting structure height varies either along the splittable outer packaging length or thickness direction.

### BRIEF DESCRIPTION OF THE DRAWINGS

A full and enabling disclosure of the present invention is set forth more particularly in the remainder of the specification, including reference to the accompanying figures, in which:

FIG. 1A illustrates a side perspective view of splittable outer packaging in accordance with the invention.

FIG. 1B illustrates a stylized cross-sectional view of the splittable outer packaging of FIG. 1A, taken from the view of directional line 1B-1B, showing the length and height directions of the splittable outer packaging. The cross-sectional view is with the front face panel 12 removed.

FIG. 1C (1-4) illustrates front-face panel partial views, from alternative embodiments of splittable outer packaging, highlighting alternative visual cues for identifying and/or opening the outer packaging, in accordance with the invention.

FIG. 1D(1-2) illustrates a front-face panel view of alternative embodiments of splittable outer packaging in accordance with the invention, depicting stacked absorbent articles in upper and lower packaging portions in phantom lines.

FIG. 1 E illustrates a front-face panel view of an alternative embodiment of splittable outer packaging in accordance with the invention, depicting absorbent articles in phantom line in a lower packaging portion.

FIG. 1F illustrates a side perspective view of an alternative embodiment of splittable outer packaging in accordance with the invention, having separated panel seams 25a, 25b and a removable sealing tape.

FIG. 1G illustrates a side perspective view of an alternative embodiment of splittable outer packaging in accordance with the invention, having multiple lines of perforation and a pull tab.

FIG. 1H illustrates a side perspective view of an alternative embodiment of splittable outer packaging in accordance with the invention, having perforation lines providing for exposed individual wrapper body, side-surfaces, upon splittable outer packaging separation.

FIG. 2 illustrates a side perspective view of an alternative embodiment of splittable outer packaging, in which splittable outer packaging is in the process of being opened in accordance with directional arrows, about a fold line 26.

FIG. 3 illustrates a side perspective view of an alternative embodiment of splittable outer packaging, in an opened and separated state, separated in accordance with directional arrows.

FIG. 4A illustrates a plan view (wrapper body-side view) of an exemplary individually-wrapped absorbent article (in which the article is shown in phantom lines), which is to be contained in splittable outer packaging of the invention.

FIG. 4B illustrates a stylized cross-sectional view of the individually wrapped absorbent article of FIG. 4A, taken along line 4B-4B.

FIG. 4C illustrates a bottom plan view of an exemplary absorbent article in the form of a feminine care panty liner, which may be contained within an individual wrapper and packaged within splittable outer packaging of the invention.

FIG. 5 illustrates a side perspective view of splittable outer packaging in accordance with the invention, in the process of being opened and bent along a fold line 26 according to directional arrows (but without yet separating into two discrete, shorter outer packages).

### DEFINITIONS

As used herein, the terms “comprise”, “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof. Similarly, the terms “include”, “includes”, “has” and/or “have”, and derivatives thereof, are intended to be interpreted as the word “comprise”, and are intended to be open-ended terms that specify the presence of any stated features, elements, integers, steps, or components, but do not preclude the presence or addition of one or more other features, elements, integers, steps, components, or groups thereof.

As used herein, the terms “nonwoven,” “nonwoven fabric,” and “nonowoven web” shall be interchangeable, and refer to a web having a structure of individual fibers or threads which are interlaid, but not in an identifiable manner as in a knitted fabric. Nonwoven fabrics or webs have been formed from many processes such as for example, meltblowing processes, spunbonding processes, coform processes, hydroentangling, and bonded carded web processes (such as thermal bonded carded webs or TBCW, and through-air bonded carded webs or TABCW).

Reference now will be made in detail to various embodiments of the invention, one or more examples of which are described below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. For the purposes of this application, like features may be represented by like numbers between the figures.

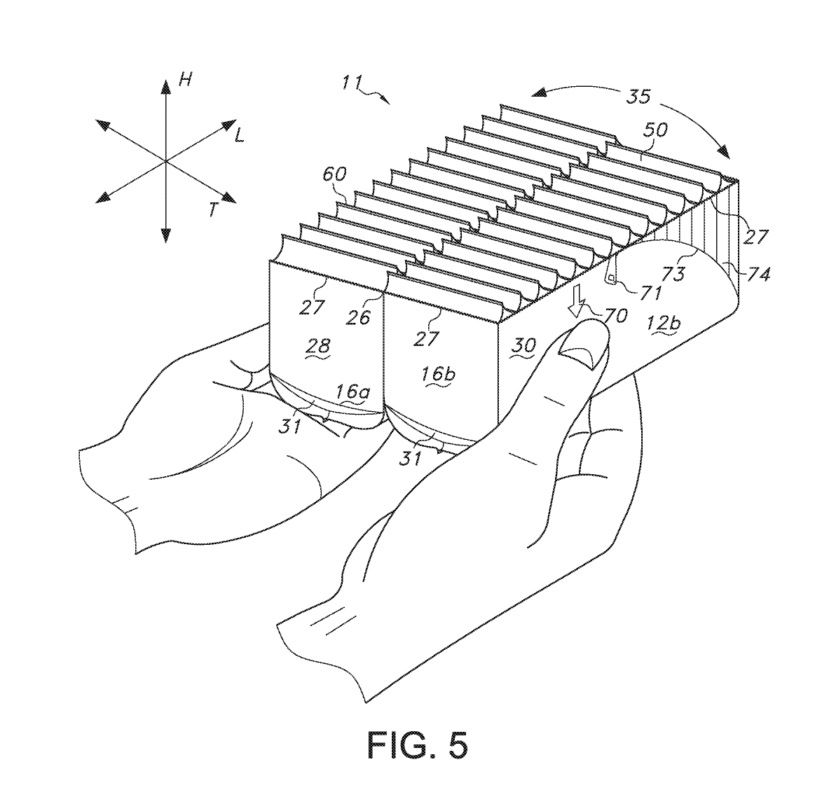

The invention is directed to a relatively large outer packaging (also referred to herein as splittable outer packaging 10) for containing individually-wrapped absorbent articles, which splittable outer packaging can be transfigured into either a single, shorter outer package 11 (FIG. 5), or alternatively, into multiple, separated and shorter outer packages 13, 15 (FIG. 3) as desired, for ease of storage by consumers. The resulting single and/or separated shorter outer packages are created by splitting the relatively large outer packaging 10 at a splitting structure (e.g. line of weakness, perforation line, or separated seams 25), on one or more of the packaging side panel(s), bending the relatively large outer packaging about a fold line 26, and then if desired, separating, by pulling apart the relatively large outer packaging 10 at the line of weakness, perforation line, or separation seams 25 (on the fold line 26), into two discrete outer packages 13, 15. The actions of splitting and bending the relatively large outer packaging about a fold line creates at least one, and in some instances depending on the embodiment, two article-dispensing openings defined by opening edges 27 in the resulting shorter package(s). The separated shorter outer packages may each be of the same size or different sizes, and may accommodate different types of absorbent articles or the same types. If the separated outer packages are of different sizes, one of the separated outer packages may accommodate a larger volume of absorbent articles than the other. The resulting shorter outer packages act as storage trays for organizing the individually-wrapped absorbent articles in relatively small numbers (compared to the original amount of individually-wrapped articles of the larger outer packaging 10), and provide for the dispensing of individually-wrapped articles through the opening(s) formed after splitting.

The individually-wrapped absorbent articles (which are each completely enveloped by a wrapper) can then be easily removed from the opening created in the transfigured outer package(s), by being grasped at an absorbent article wrapper(s), sealed edge 60. The relatively rigid wrapper sealed edges (when compared to the wrapper body portion 53) are originally positioned within the splittable outer packaging (before splitting) such that at least some are exposed at the packaging opening(s) upon splitting of the packaging. In some embodiments, each wrapper includes at least one sealed edge that is positioned at the exposed openings following splitting. The sealed edges 60 act as easily identifiable handles to withdraw the articles. Such article removal can be either one-at-a-time, or in multiples as desired, and without risk of incidental soiling of the absorbent article itself. Since the splittable outer packaging 10 may be stored in its original, relatively large format in an under-cabinet or closet, or alternatively, as a single shorter outer packaging having two separated openings for article removal (such as for storage in a short drawer), or even as multiple shorter outer packages each having an opening, (such as for storage in two separate short drawers), the splittable outer packaging 10 is adaptable to accommodate the various storage needs of a consumer (and the various height restrictions of secondary storage containers).

The splittable outer packaging is desirably fashioned from a flexible polymeric film or metallic, foil-based material, but may alternatively be made from paper, cardboard, or more rigid polymer components, such as molded plastic materials as are known in the art. In one embodiment, the splittable outer packaging is fashioned from a polyolefin film, such as a polyethylene (PE), polypropylene (PP), polymethylpentene, polybutene-1, polyolefin elastomers, polyisobutylene, ethylene propylene rubber (EPR), ethylene propylene diene monomer (M-class) rubber (EPDM rubber), or alternatively, from rayon or nylon, the film having a basis weight in one embodiment of between about 18 and 280 grams per square meter (gsm), alternatively, between about 40 and 60 gsm.

If such splittable outer packaging is formed from molded plastic materials, it may for example, be formed from polypropylene (PP), polyethylene (PE), polystyrene, polyethylene terephthalate (PET), high density polyethylene (HDPE), low density polyethylene (LDPE), polyvinyl chloride (PVC), polyester, polyvinylidene chloride (PVDC), high impact polystyrene (HIPS), polyamides (PA), acrylonitrile butadiene styrene (ABS), polyethylene/acrylonitrile butadiene styrene (PE/ABS), polycarbonate (PC), polycarbonate/acrylonitrile butadiene styrene (PC/ABS), polyurethane (PU). Molded storage material can also be made from thermoplastic polyurethane (TPU), rubber, and silicones. Further splittable outer packaging may be made from molded paper. In yet a further alternative embodiment, the splittable outer packaging may be formed from metalized polyfilms, such as metalized PET film and LDPE, and metalized PP film and LDPE.

If such splittable outer packaging is formed from cardstock/paperboard, it is desirable in one embodiment for the cardstock to be between about 0.014 inch (0.04 cm) to about 0.024 inch (0.06 cm), alternatively, about 0.016 inch board grade (0.04 cm). Such paperboard may include SBS/duplex board/coated/laminated) having a basis weight of between about 45 gsm and 800 gsm, alternatively between about 180 gsm and 400 gsm.

The individual absorbent article wrappers 50, which completely envelop the absorbent articles 52, are desirably fashioned from film, nonwoven sheets, paper or tissue sheets, or laminates thereof, as are also known in the art. Alternatively, such wrappers may be formed from sealed or coated paper, mesh, or netting, having sealed edges. In one embodiment, such wrappers are formed from a spunbond-meltblown-spunbond nonwoven laminate sheet, having a basis weight of between about 15 and 30 gsm, alternatively, between about 15 and 24 gsm. In a further embodiment, such wrappers are formed from a polyolefin (e.g. polyethylene) film having a basis weight of between about 18 and 30 gsm, alternatively, between about 18 and 25 gsm. In one embodiment, such splittable outer packaging 10 and individual wrappers 50 are both formed from the same material. In a further embodiment, they are formed from different materials, such as the splittable outer packaging 10 being formed from film, while the wrappers 50 being formed from nonwoven laminates.

If such splittable outer packaging 10 and wrappers 50 are manufactured from a film sheet, it may be desirably in one embodiment, to utilize film materials that provide a kinetic coefficient of friction (COF) that avoids processing and packing issues resulting from film slippage, and that also encourages relatively easy slipping of adjacent film surfaces within the outer packaging. For example, kinetic COF values (as determined using ASTM 1894-14) may be selected to discourage the slipping of film sheets during processing (and consequential expulsion of wrapped articles from stacks during processing). While at the same time, values may be selected for at least the wrapper body portions 53 that facilitate the easy removal of adjacent wrapped articles from the split outer packages 11, 13, 15, and from other adjacent wrapped articles.

For example in one embodiment, the individual wrappers (outside wrapper surfaces) may demonstrate a range of kinetic COF of between about 0.1 to 1.16 or higher, alternatively, between about 0.1 and 0.7, alternatively, between about 0.1 and 0.40, alternatively between about 0.1 and 0.25. Values may be similar for the outer packaging. In an alternative embodiment, the wrapper COF may be higher than that of the outer package. In a further alternative embodiment, wrappers may include zones along their surfaces that demonstrate different COF, in order to prevent inadvertent sliding of articles out of the outer package once opened. In an alternative embodiment, the sealed edges 60 of the wrappers 50 may demonstrate a kinetic COF that is higher than that of the wrapper body portion 53 to allow for easier grasping of the individual wrapper. Further examples of wrappers with relatively low kinetic COF are described in European Patent No. 1941852B1 to Toro et al., which subject matter is incorporated by reference hereto in its entirety, to the extent not inconsistent with the subject matter of this disclosure. The individual wrapper surface friction (kinetic COF) can be controlled by either the selection of wrapper materials themselves or surface treatments. The control of the friction level may specifically be used to increase both the ease by which wrappers are able to smoothly slide from the outer package, as well as the unintended ejection out from the outer packaging while it is being split open.

The wrappers are desirably sealed at least at one edge, but alternatively at two opposing edges, such as by the use of ultrasonic or thermal bonding. In a further alternative, such wrappers are sealed at three, or alternatively, all four edges (of a square or rectangularly-shaped pouch/wrapper). At least some amount/portion of the sealed edges of the individual wrappers are aligned within the splittable outer packaging such that they are exposed at openings formed upon the splitting of the splittable outer packaging.

The individually-wrapped absorbent articles are desirably folded personal care absorbent articles that are used to collect body exudates (i.e. menses or urine), such as for example, folded feminine care hygiene pads, napkins, or liners, or adult care incontinence products, such as undergarment inserts, pads, garment shields, or absorbent pants. The relatively large outer packaging 10 may contain a single type of individually-wrapped absorbent article, or multiple types of individually-wrapped absorbent articles, such as for example, a combination of pads and panty liners, a combination of overnight-style feminine hygiene pads and daily-use feminine hygiene pads, or absorbent inserts or shields, and absorbent pants. For the purpose of this application, the term “overnight-style feminine hygiene pad” refers to a sanitary pad designed for use over an extended time period, such as throughout a night while a consumer is sleeping. This is in contrast to a daily-use feminine hygiene pad product which is typically worn by a consumer for a relatively shorter time span and replaced throughout the day.

Alternatively, such splittable outer packaging 10 may include only one type of absorbent articles, but instead, articles differing only in performance attributes, aesthetic details, or length. For example, the splittable outer packaging may include individually wrapped, multiple-sized pads, pads having the same size but different levels of absorbency, or other physical attributes such as scented and unscented versions, for use in response to a consumer's particular daily needs, mood, daily aesthetic preference, volume of menses flow, or incontinence levels.

As noted, the relatively large outer packaging 10 includes a structural feature (splitting structure 25) for physically separating two connected outer packaging portions either partially, or completely into at least two discrete portions (or partially separated portions), such as for example, a perforation line, a line of weakness, or physically separated panel seams, which separated seams are held together by a fastening device until the shorter outer packaging size is needed. In an alternative embodiment, the splitting structure 25 is designed for both visual and physical separation of two connected packaging portions 28, 30.

For example, such separated seam embodiment may be covered by a sealing and removable ribbon or tape which connects the two portions, and acts as a fastening device. Alternatively, such separated seam embodiment may be held together by a further outer wrapping material, a tie-around ribbon, or cord (not shown). In still a further alternative embodiment, if such splittable outer packaging is fashioned as a box-like enclosure (such as from a more rigid polymeric, wood, metal, or cardboard-based material), such box may be separated via the sliding or rotational movement (i.e. screw-on top) of the fitted/interlocking box lid and body, or box halves.

The process of transfiguring (i.e. splitting, bending, and optionally separating) the relatively large outer packaging (such as of a flexible sheet material) into a single, relatively shorter outer package, or multiple, relatively shorter outer packages, results in at least the partial separation of two or more portions of the relatively large outer packaging (and in particular, at least three adjacent face panels in a six-sided package) at a splitting structure 25. The partially separated portions of the relatively large outer packaging each include an easy, article-removal feature. Specifically, at least a portion of individually-wrapped absorbent articles are accessible, easily identifiable, and easy to grab for removal from the resulting shorter, and open outer package by their exposed sealed edges.

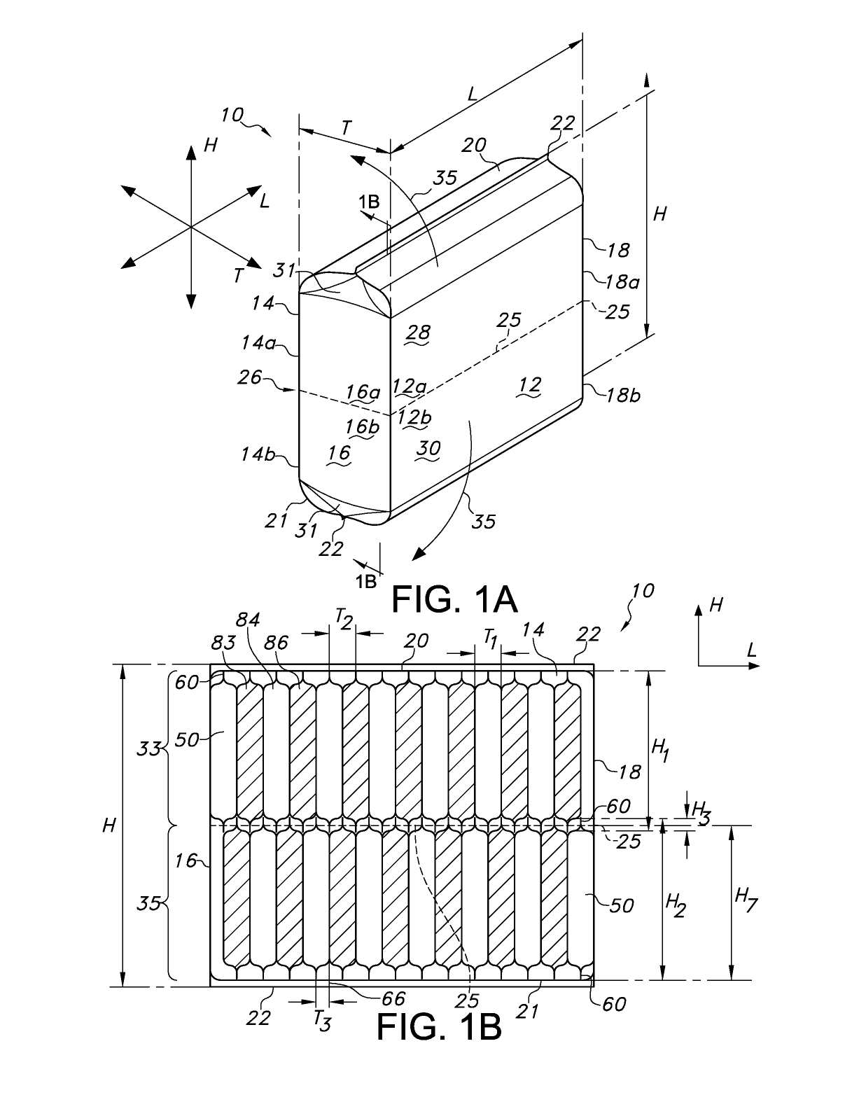

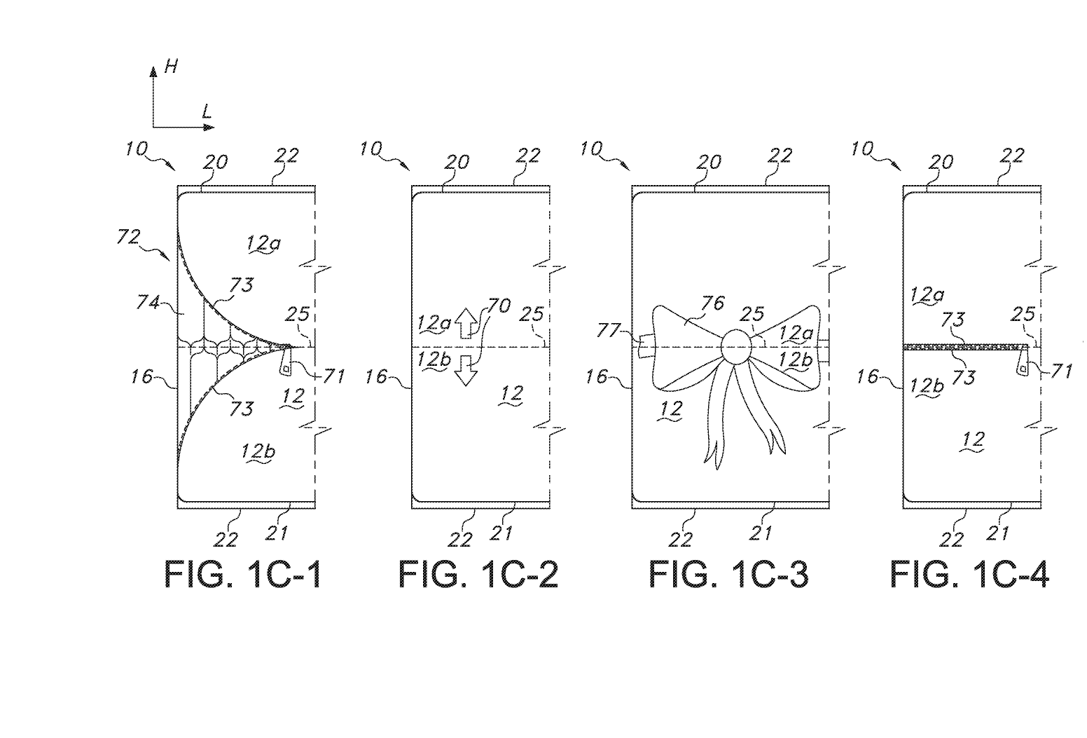

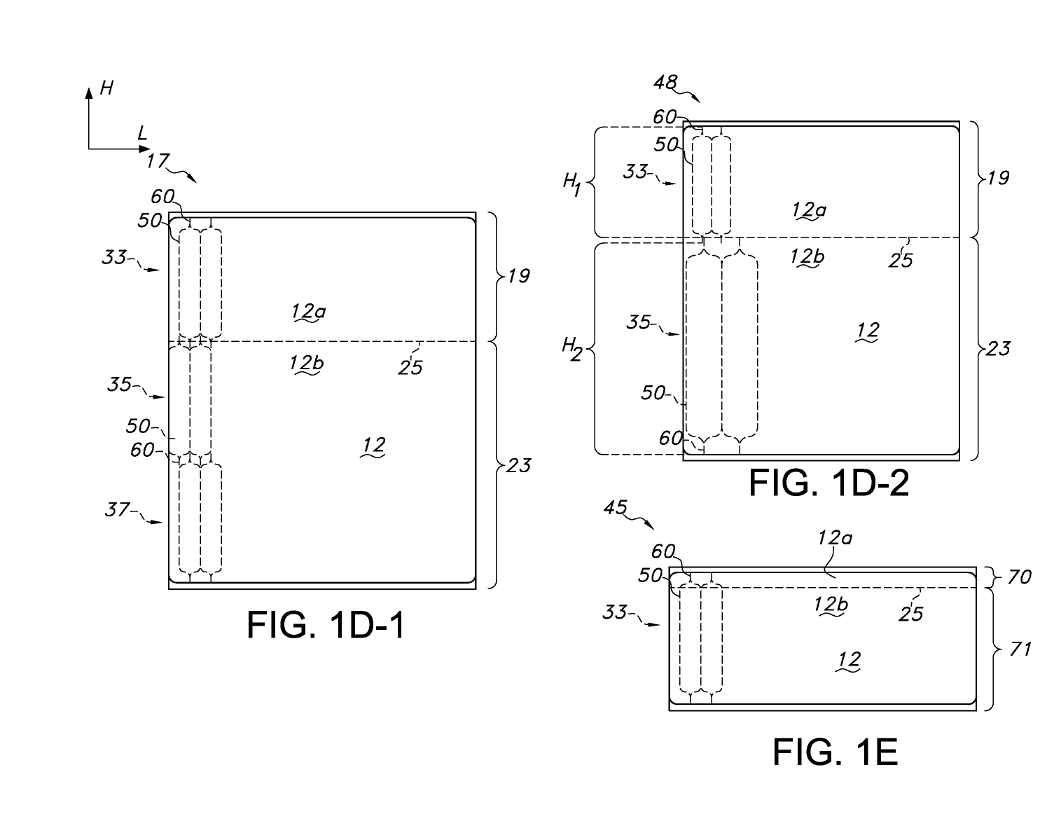

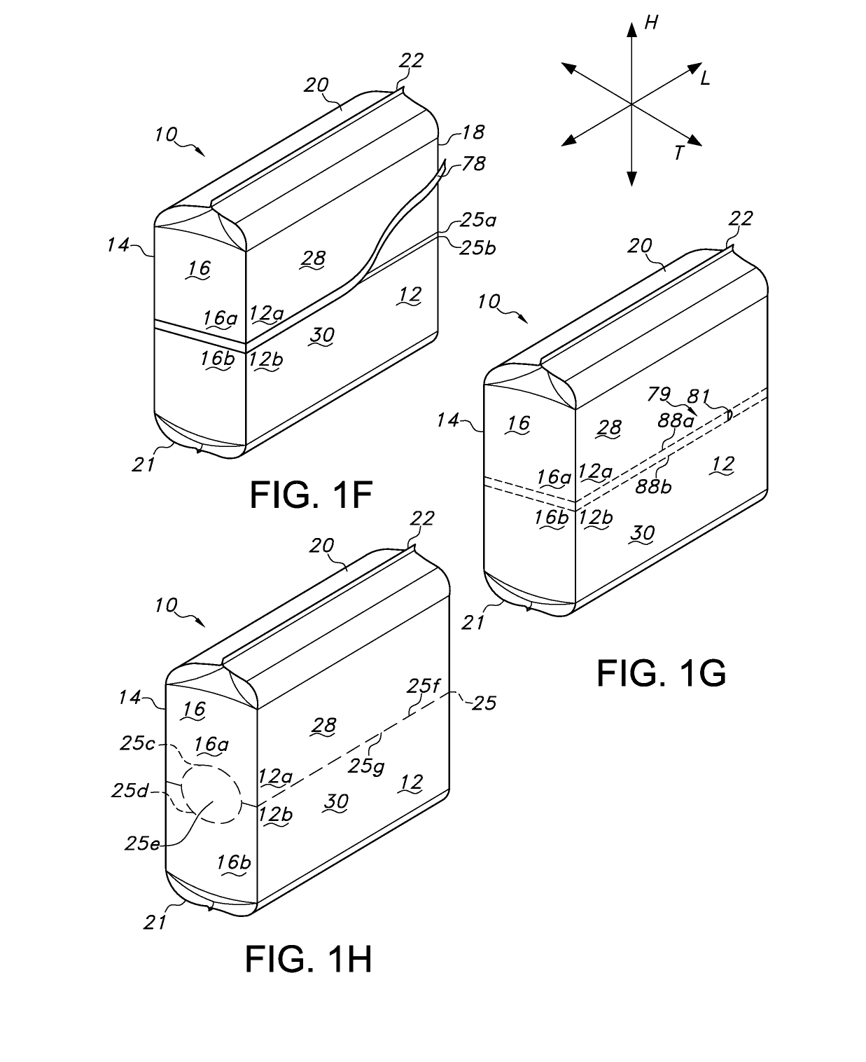

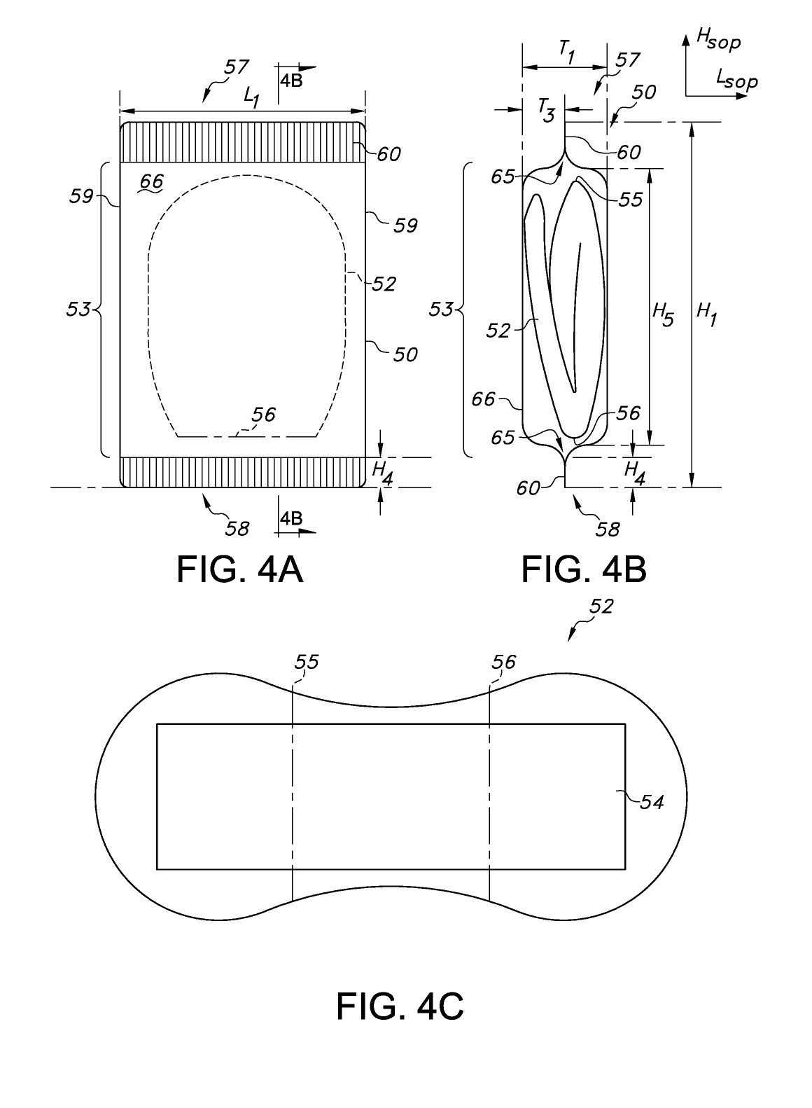

In accordance with the invention, a specific example of splittable outer packaging 10 containing individual wrappers 50 (i.e. inner packaging which each contain a single, folded absorbent article), is illustrated in FIGS. 1A and 1B. A side perspective view is shown in FIG. 1A, while a cross-sectional view is shown in FIG. 1B. The splittable outer packaging 10 has a length direction L, a height direction H, and a thickness direction T. For the purpose of ease of reference in this disclosure, the individually-wrapped personal care absorbent articles are all illustrated as wrapped feminine care panty liners (shown in detail in FIGS. 4A-4C). It should be understood however, that while only panty liners are illustrated in this disclosure, many types of personal care absorbent articles as previously described, are contemplated to be within the scope of the invention. In a desirable embodiment, foldable personal care absorbent articles that may be retained in compressed stacks, are stored within the splittable outer packaging 10 of the invention.

A plan view of an individual wrapper 50 to be contained (in at least one stack of multiple wrapped articles) within splittable outer packaging 10 and with each wrapper including a folded feminine care panty liner 52 as the personal care absorbent article (in phantom line), is shown in FIG. 4A. The individual wrapper 50 completely envelops the personal care absorbent article 52 on all sides, such that the article will not be directly exposed to the surrounding environment unless the wrapper 50 is ruptured in some fashion. The individual wrapper 50 includes a wrapper body portion 53 and at least one sealed edge portion 60. The wrapper body portion 53 has relatively large planar side edges 66. The size of the side edges 66 will depend on the overall size of the absorbent article and the number of folds in the absorbent article. The particular feminine care panty liner 52 illustrated, is of a relatively small size with limited numbers of folded edges.

Folded feminine care panty liners 52 for placement in the crotch region of a consumer's undergarment, typically include a liquid permeable topsheet layer, a liquid impermeable backsheet layer, and an absorbent core layer sandwiched there-between (all not shown, but are generally known in the absorbent article art). The panty liner 52 also typically includes garment attachment adhesive (not shown) that is covered with a protective release sheet 54 until the article is to be used (FIG. 4C). The exemplary panty liner 52 includes two fold lines 55, 56, although one or more numerous fold lines are also contemplated. The folded panty liner 52 is desirably placed within the wrapper 50 such that the folded edges of the panty liner 52 are either generally parallel with the length L1 of the sealed edge 60 of the wrapper (as seen in FIGS. 4A) or generally perpendicular to the length direction L1 of a sealed edge 60 (not shown).

The wrapper 50, has a wrapper length L1, a wrapper thickness T1 (encompassing the wrapper and the folded article), and a wrapper total height H1 (FIGS. 4B). The height and length directional arrows shown in association with FIG. 4B are representative of the height and length directions within the splittable outer packaging 10 FIG. 1, hence the abbreviations “sop”. The wrapper body portion 53 has a wrapper body portion height H5. The at least one sealed edge 60 has a sealed edge height H4. Desirably in one embodiment, the wrapper total height H1 is larger than the height of the splitting structure H7 of the splittable outer packaging 10 (the distance of the splitting structure 25 from the internal package surface of the bottom panel 21 of the splittable outer packaging 10) , such that following splitting of the splittable outer packaging 10, the wrapper sealed edges protrude from the resulting outer package and above the resulting outer package opening edge 27. The wrapper body portion 53 generally surrounds the panty liner 52 and is often in direct contact with panty liner surfaces while the panty liner is stored in the wrapper 50. The at least one sealed edge 60, desirably extends outward from the wrapper body portion and away from the panty liner 52. The sealed edge(s) 60 may also be bent in storage (not shown) to lie one over the other or in physical contact with a vertically adjacent sealed wrapper edge, or may desirably stand outwardly or vertically outwardly at the ends 57, 58 of the wrapper 50 as illustrated in FIGS. 4A, 4B. The portion of the sealed edge 60 that is inside 65 of the wrapper 65, may or may not come in contact with the panty liner 52 while it is stored in the wrapper 50.

In the illustrated embodiment, the wrapper 50 includes two opposing sealed edges 60. While the opposing sealed edges 60 are illustrated as having approximately the same height H4, they need not. Further, as noted, the wrapper may include only one sealed edge 60, three sealed edges, or four sealed edges. The sealed edges 60 may encompass an extended length portion of the wrapper or a seal line at the wrapper end(s) 57, 58. Within the splittable outer packaging 10, the length of the wrapper L1, and consequently the at least one sealed wrapper edge 60, are in one embodiment, both parallel with the thickness direction T of the splittable outer packaging 10. In an alternative embodiment, they are perpendicular to the thickness direction T. It is desirable in one embodiment, for the thickness direction measurement of the splittable outer packaging 10 to be less than the length direction L measurement.

The wrapper body portion 53 has a height H5, which is the height of that portion of the wrapper 50 exclusive of the sealed edge 60, such as between the sealed edges 60 (if two), or desirably in one embodiment, the wrapper portion which most directly encloses the absorbent article 52, exclusive of the sealed edge(s). A thickness distance T2 separates adjacent sealed edges 60 within the splittable outer packaging 10 (FIG. 1B). The wrapper has a partial thickness T3 which extends from an imaginary line through the sealed edge 60 to the outer edge 66 of the wrapper body portion 53. The wrappers 50 are desirably folded sheets that completely envelop the folded absorbent article 52, and that are then sealed at least at one, but desirably at two or more wrapper ends 57, 58 to form the sealed edges 60. In one such embodiment, the wrappers are sealed at two opposing ends 57, 58 as shown in FIG. 4A. The remaining wrapper edges/ends 59 may be comprised of folded wrapper portions or additional seals. Such sealing (at the sealed edges 60, other edges/ends 59, or inboard of the other edges/ends 59 along the side edges 66, may be accomplished by thermal, adhesive, ultrasonic, pressure, needlepunching/stitching, staple, or other generally known bonding techniques, or combinations thereof.

The sealed edges 60 of the individually-wrapped absorbent articles may in one embodiment, include different or additional construction materials from the wrapper body portions 53 (such as additional film or nonwoven strips only at the sealed edge 60), different surface textures from the wrapper body portion 53, or different visually apparent features from the wrapper body portion 53, to aid the consumer in identifying individual absorbent articles in the outer package, types of absorbent articles, or a specific number of multiple absorbent articles within the split outer package 11,13,15 that are to be removed for use. Additional construction material such as adhesive, film, or nonwoven strips may be present only at the wrapper sealed edges 60 to provide for reinforcement or strengthening of the sealed edges 60 for ease of grasping. Additional rigidity at the wrapper sealed edges 60 provided by additional wrapper construction material can assist the consumer in withdrawing a single individually-wrapped absorbent article from the transfigured outer package. Such rigidity can also be created by use of high density bonding processes such as high density ultrasonic or thermal bonding patterns (as are known in the bonding art) only at the sealed edges 60. Further, by providing adequate spacing T2 between exposed (and optionally immediately adjacent) sealed wrapper edges 60 within a split outer package 11,13,15, wrappers 50 may more easily be identified and withdrawn from the stack or row of absorbent articles contained in the transfigured (shorter) outer package(s). Therefore, in one embodiment, only a portion of the total wrapper sealed edges are exposed adjacent the outer package opening edge 27 (not shown).

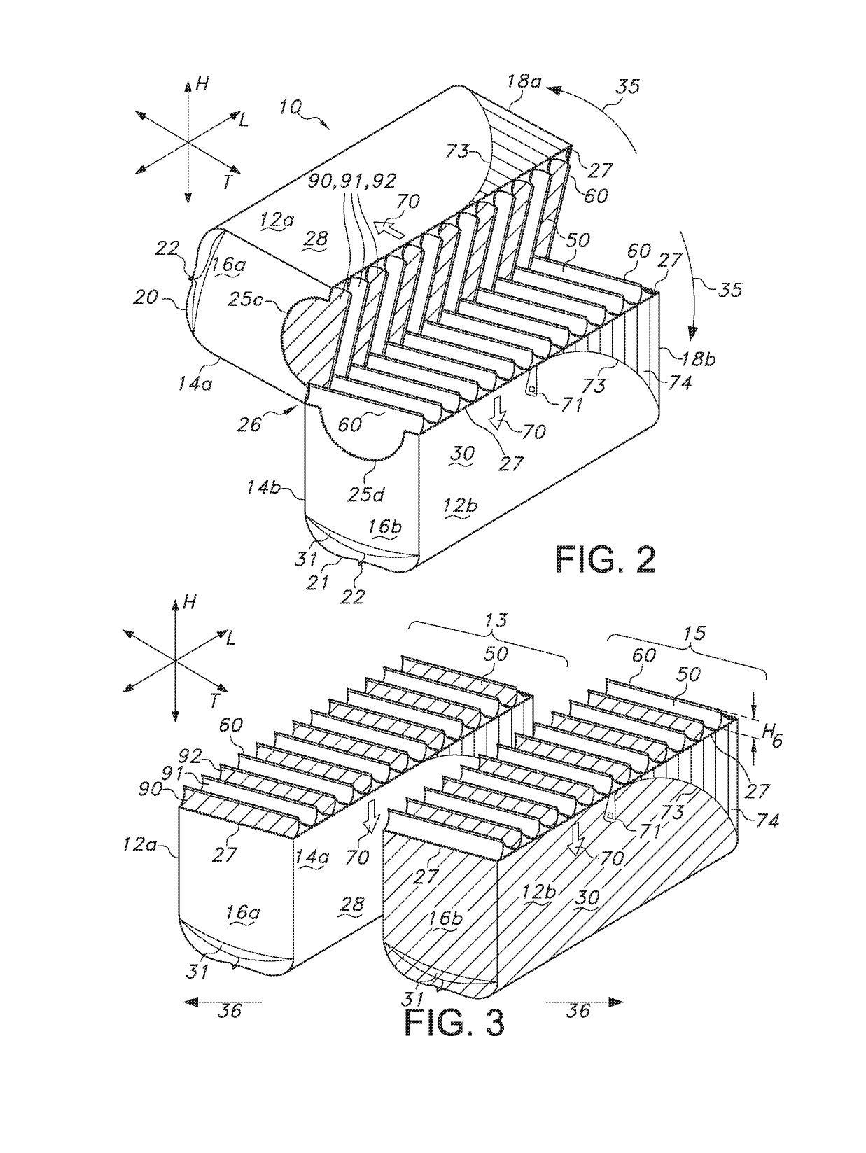

In still a further alternative embodiment, only the sealed edges 60 themselves of adjacent, individually-wrapped absorbent articles may be differently or alternately colored, textured, or constructed to help consumers in selecting and removing the desired type, or amount of absorbent articles. For example, the splittable outer packaging 10 may contain different types of individually-wrapped absorbent articles, and the visually distinguishable wrappers 50 and/or sealed edges 60 can provide assistance to the consumer in identifying the particular type of absorbent article desired. As an example, differently colored sealed edges 60 or wrappers 50 can be used to identify absorbent articles that differ in absorbency levels or sizes, which were originally contained within the same splittable outer packaging 10, but which, after the transfiguring of the large outer packaging, are concentrated in separated small outer packages (as seen in FIG. 2, at 90, 92). Without such visual indicators, such different absorbent articles may be otherwise difficult to distinguish. Such differently colored wrappers are also shown as 83, 84, 86 in FIG. 1B. Alternatively, the coloration of the relatively large splittable outer packaging 10 may be such that upon splitting and separation, each of the partially separated shorter package portions 11 or completely separated small packages 13,15 have a different primary outer package color (as seen in FIG. 3 in which portion 30 is shown of a different color than portion 28). Such color differences can further assist consumers in selecting the absorbent article most suitable for their daily needs, or placement of specific shorter outer packages with different articles, in separate locations.

In a further embodiment, the individually wrapped articles may be alternately placed within the splittable outer packaging such that only every other individual article wrapper sealed edge is facing the opening edge 27 (not shown) upon outer packaging splitting. In such a fashion, more space will be available for the consumer to easily grasp individual wrapper sealed edges 60 from within the resulting shorter package(s). Once such wrappers with sealed edges facing the opening are removed first, removal of the remaining wrappers in such embodiment (without sealed edges facing the opening 27) can be more easily accomplished.

In yet still a further alternative embodiment, immediately adjacent individually wrapped absorbent articles may be wrapped in visually distinguishable wrappers (wrapper body 50 and/or sealed edges 60), so as to facilitate their removal from the shorter outer packages (as seen in FIG. 1B at 83, 84, and 86 and FIGS. 2, 3 at 90, 91, and 92). For example, some individually-wrapped articles may be contained in film-based outer wrappers, while others are contained in fibrous nonwoven outer wrappers, as long as at least some sealed wrapper edges 60 of each are oriented in the same direction facing the outer package opening (following splitting and at least partial outer package separation). Such alternating wrapped articles may be distinguished by color as previously noted.

As noted, a side perspective view of splittable outer packaging 10 in accordance with the invention is shown in FIG. 1. As seen in the figure, the packaging generally has an outer packaging length direction L, an outer package height direction H (which is actually an “initial” outer package height), and an outer package thickness direction T. The splittable outer packaging 10 has an “initial” height because following at least partial separation, the resulting partially or completely separated outer package(s) will have at least one shorter height (or “final” height) when compared to the initial height of the splittable outer packaging 10. This shorter height may correspond to the height H7 of the splitting structure 25 (especially when the splitting structure 25 was originally positioned midway along the splittable outer packaging height direction H). In some embodiments, the resulting split packages will have two different final, but shorter heights (when the splitting structure 25 was not originally positioned midway along the splittable outer packaging height direction H).

The splittable outer packaging 10 shown in FIG. 1 includes, a frontside face panel 12, a backside face panel 14, a first side face panel 16, a second side face panel 18, a top face panel 20 and a bottom face panel 21. Depending on actual splittable outer packaging design and construction materials, the splittable outer packaging 10 optionally includes a top and/or bottom face bond seam 22 extending along the outer packaging length direction L, for holding the splittable outer packaging 10 together. For example, such bond seam(s) 22 may not be necessary, depending on the outer packaging forming process or forming materials (such as if paperboard, cardboard, metal, wood, or molded plastic are used in lieu of film or foil). While the splittable outer packaging 10 is desirably formed from a flexible sheet material, such as a polymeric film, metallic foil, a polymeric nonwoven sheet material, or a laminate of any of the foregoing film, foil, and polymeric nonwoven sheet materials as are generally known in the art, it may also desirably be constructed of paper, coated paper, paperboard, or cardboard materials as are also generally known in the art. While not desired, the bond seams 22 may instead be positioned along the side face, or front and back face panels.

While shown in FIG. 1 as having generally six faces (with four side face panels), it should be appreciated that such splittable outer packaging 10 may instead, be of a cylindrical or other geometric, three-dimensional shape (not shown). For example, the splittable outer packaging 10 should have at least one surface that functions as a side face panel (i.e. at least one side face panel), which side face panel can be split into two separated portions along its height. For instance, a cylindrically-shaped splittable outer packaging of the invention would have one side face panel that connected a top and bottom face panel, and which one side face panel would include a splitting structure 25 along the one side face panel (not shown), which circumscribes the tubular side face panel. The particular splittable outer packaging 10 of FIG. 1, is shown having optional folds or gusset-like features 31 on each of the first and second side face panels 16, 18.

The splittable outer packaging 10 is desirably splittable (meaning, at least partially separable) as a result of the inclusion of a splitting structure 25. The splitting structure in one embodiment, may be either a line of weakness, or a perforation line 25 which extends across at least three immediately adjacent side face panels of the splittable outer packaging 10. Desirably, the perforation line 25 is placed on at least the frontside face 12 and two side face 16, 18 panels. For example, at least one continuous line of weakness or perforation line 25 may be placed within the frontside face panel 12, the first side face panel 16, and the second side face panel 18. Alternatively, the line of weakness or perforation line 25 may be placed continuously between four adjacent side face panels (12, 14, 16, 18), such that it circumscribes the entire splittable outer packaging 10 (desirably in one embodiment, at the same height H7 (as seen in FIG. 1 B) in each front, back, and side face panel, measured from the inside surface of the bottom face panel 21 of the splittable outer packaging 10). The splitting structure 25 is desirably located at a height H7 along the at least one side face panel of the splittable outer packaging 10 (as seen in FIG. 1 B) that enables the split packaging (11, 13, 15) to fit within relatively short drawers after package splitting. The height H7 may be uniform along the at least one side face panel of the splittable outer packaging 10 as noted, or it may vary along the packaging length direction L. It may also vary along the packaging thickness direction T as discussed below with respect to FIG. 1H. Desirably, in one embodiment, the height H7 is between about 40 mm (such as for liners) and 250 mm (such as for multiple stacks or incontinence articles), alternatively, between about 40 and 100 mm.

The splittable outer packaging 10 may be grasped at the front 12 and back side face 14 panels, and with a slight outwardly bending motion of a consumer's hands along the direction of arrows 35, the splittable outer packaging 10 may be popped open along the line of weakness or perforation line 25, thereby at least partially splitting the packaging along at least three side faces (and in some instances as desired, four side faces). The splittable outer packaging is in one embodiment, folded or bent open along fold line 26 (which may also be the previously described line of weakness or perforation line 25 if present). The action of folding-open the splittable outer packaging 10 creates two openings in the resulting single shorter package 11 (seen in FIG. 5). The openings are defined by opening edge 27. The resulting shorter package 11 may also be pulled apart (along directional arrows 36) so as to form two discrete, relatively shorter packages (seen in FIG. 3), each having an opening defined by an opening edge 27. Desirably in one embodiment, the width (or thickness direction of the opening) is approximately the same size or slightly larger than the L1 dimension of the individual wrappers 50. By “approximately” is meant within about 10 mm, alternatively within about 5 mm, alternatively, within about 1 mm, alternatively, about the same size as the size of the L1 dimension of the wrappers 50. Such similar opening and wrapper dimensions allow for less problematic retrieval of individually-wrapped absorbent articles from the transfigured outer packaging. In one embodiment, the size of the L1 dimension of the wrapper 50 (and thickness direction T of the splittable outer packaging 10) is desirably between about 30 mm (such as for liners) and 200 mm (such as for incontinence articles), alternatively, between about 50 mm and 150 mm, alternatively, between about 70 mm (such as for liners) and 110 mm (such as for pads). Smaller L1 and outer package thickness direction dimensions (such as less than 80 mm) are useful, in that consumers avoid having to open their hands to the maximum extent, in order to grasp the outer packaging while splitting it open.

The line of weakness or perforation line 25 may be formed by any material weakening or perforating techniques known in the art, such as by embossing methods, aperturing/punching methods, cutting methods, or a combination thereof. For example, such line of weakness/perforation line 25 may be created by a thermal and/or pressure embossing technique which melts or thins portions of the package, or laser cutting/knife aperturing, which cuts or punctures portions of the package. While it is desirable that such splitting structure 25 be a generally straight line of weakness or perforation line, wavy or other shaped splitting structures are also contemplated, such that the opening edge 27 in the resulting split package(s) are more ornamental in appearance, or provide for better viewing of wrapper body portions 53 after the large outer packaging is split. Desirably in one embodiment, the ratio of area of perforated or embossed regions or dashes 25f (in the case of a perforation line or line of weakness respectively, such as seen in FIG. 1H) to the space of solid film or other outer packaging material 25g between the perforated or embossed dashes, is between about 0.5 and 0.7. It has been found that such a ratio of perforated or embossed regions to nonperforated or nonembossed regions, allows for the splittable outer packaging 10 to be separated neatly with a relatively smooth outwardly directed motion of a consumer's hands. The resulting opening edge 27 is also fairly uniform in appearance. It has been determined that if the ratio is much higher, the splittable outer packaging 10 risks being inadvertently opened or damaged. If the ratio is much less than the noted values, the splitting structure 25 becomes difficult to see, the splittable outer packaging 10 becomes more challenging to open easily, and such splitting may result in an irregular opening edge 27.

As seen in either of the embodiments illustrated in FIGS. 1B, 1D, and 1E, the splittable outer packaging 10, 17, 45, 48 respectively, contain numerous pouch-like wrappers 50, which individual wrappers each envelop a single absorbent article (panty liner) 52. The individually-wrapped absorbent articles are present in the packaging 10 in at least one stack of articles 33 (that may be optionally compressed to accommodate more articles in each stack). A single stack of individually-wrapped absorbent articles is shown in phantom in FIG. 1E. In the embodiment of FIG. 1E, the splittable outer packaging 45 includes an upper 70 and lower portion 71 that may be separated at a splitting structure (e.g. perforation line) 25 such that the bottom portion 71 is the only portion which contains wrapped articles after package splitting. In some contemplated embodiments, the splittable outer packaging 10, may include multiple side-by-side stacks of individually wrapped absorbent articles, 33, 35, 37 as seen for example in FIGS. 1B and 1D. Two parallel stacks are shown in the cross-sectional view of FIG. 1B. Three parallel stacks of individually-wrapped absorbent articles are shown in phantom in FIGS. 1D-1). The line of weakness or perforation line 25 divides the package into asymmetrically sized upper and lower portions 19, 23, which upon splitting, results in the creation of two asymmetrically shaped (of unequal heights) outer packages. The splitting of the outer package embodiment 48 of FIG. 1D-2 results in the placement of larger volumes of smaller, wrapped panty liners (in phantom) from one stack 33 into one outer package, and smaller volumes of larger wrapped pads (in phantom) from the other stack 35 into a second outer package.

In the embodiment of FIG. 1A, the splitting structure (line of weakness or perforation line 25) acts to physically and optionally visually, separate the frontside, side (one or two), and potentially backside face panels into two subpanels each. For example, the perforation line 25 separates the frontside face panel 12 into an upper, frontside face subpanel 12a and a lower, frontside face subpanel 12b. Similarly, the perforation line 25, if present on the backside face panel 14, separates the backside face panel into an upper, backside face subpanel 14a, and a lower backside face subpanel 14b. The perforation line 25 also separates the first side face panel 16 into an upper, first side face subpanel 16a, and a lower, first side face subpanel 16b. The perforation line 25 also separates the second side face panel 18 into an upper, second side face subpanel 18a, and a lower, second side face subpanel 18b.

As a result of the line of weakness or perforation line 25, the relatively large splittable outer packaging 10 of FIG. 1, includes the upper portion 28 (defined by the upper, side face subpanels 12a, 14a, 16a, 18a, and top face panel 20) and the lower portion 30 (defined by the lower, side face subpanels 12b, 14b, 16b, 18b, and bottom face panel 21) prior to the act of splitting the packaging 10. The upper and lower portions 28, 30 of the relatively large splittable outer packaging 10 are initially connected (either directly or indirectly). They also eventually form the connected, but open portions of the shorter outer packaging 11 (of FIG. 5), or alternatively the separated, relatively small outer packages 13, 15 (of FIG. 3) with two separated openings as well.

Whether the line of weakness or perforation line 25 extends completely around the splittable outer packaging 10, 17, 45, the act of at least partially separating the portions along the line of weakness 25 (on three face panels) allows the relatively large splittable outer packaging 10 to bend and open in the direction of the arrows 35 such that the outer packaging can bend open along fold line 26. Depending on whether fold line 26 is also a line of weakness or perforation line, it may also allow for the full separation of the backside face panel 14. Therefore, at the option of the consumer, and depending on the presence or absence of a line of weakness or perforation line on the backside face panel 14, the fold line 26 may also allow for separation of the backside face two subpanels 14a, 14b and therefore, the complete separation of the large outer package portions 28 and 30, into two discrete, smaller outer packages 13, 15.

By placement of the line of weakness or perforation line 25 in three immediately adjacent side face panels, the large splittable outer packaging 10 may be transfigured into an open, but shorter outer packaging configuration 11 (when compared with the height of the original splittable outer packaging 10), once it is opened about fold line 26 (as seen in FIG. 5). Alternatively, the line of weakness or perforation line 25 may be present in four side face panels, but only opened along three of the side face panels to produce the shorter single package. Whether the large splittable outer packaging 10 is split to produce a single but shorter outer package 11, or separated into two discrete shorter outer packages 13, 15 (when compared to the dimensions of the starting package), the resulting outer packages may then both be capable of fitting into a shorter secondary container, such as a bathroom cabinet drawer, a cabinet shelf, or discrete, office work-space. If the large splittable outer packaging 10 is split into shorter, separated outer packages 13, 15, the separated outer packages may also be placed in two physically separated locations within a consumer's home or office environment.

The two portions 28, 30 of the relatively large splittable outer packaging 10, may be of generally equal height, width, and length as shown in FIG. 1A, 1B, such that the partially separated (as seen in FIG. 5), or totally separated portions (as seen in FIG. 3) are each half of the splittable outer packaging 10, capable of housing the same number of individually wrapped absorbent articles if the splittable outer packaging 10 contains the same type of absorbent articles in each stack of articles. In such situation, the line of weakness or perforation line 25 is desirably positioned midway along the large splittable outer packaging 10 height direction H. Such height H7, is equidistant between the top and bottom face panels (inside surfaces).

Such perforation line 25, as shown in FIG. 1B, is desirably in one embodiment, positioned along the package height direction H such that sealed edges 60 of the wrappers 50 contained within the large splittable outer packaging 10 are aligned with and adjacent to the position of the perforation line 25. That is, upon splitting and bending the splittable outer packaging 10 open, the wrapper sealed edges 60 in each split outer package are approximately at the same height as the height of the opening edge 27 on the resulting shorter packages, 11, 13, 15, or extend slightly above the opening edge 27 as seen in the figures. For example, it is desirable in one embodiment, for the wrapper sealed edges 60 to extend between about 4 mm (such as for liners) and 10 mm (such as for pads), alternatively between about 5 mm (such as for liners) and 8 mm (such as for pads) above the height of the opening edge 27 following the splitting, opening, and bending (and separation as the case may be) of the outer package into the shorter outer package format. This distance is illustrated by H6 in FIG. 3. Alternatively, the wrapper height (including sealed edge portions 60 and wrapper body portions 53) may extend above the opening edge 27 of split outer packages (11, 13, 15), by between about 4 and 30 mm, alternatively, by an amount less than 30 mm. Alternatively, in one embodiment, the sealed edges 60 are lower than the opening edge 27 following splitting, opening, and bending (and separation), such as shorter by between about 4 mm and 10 mm (all measurements when the shorter outer packages 11, 13, and 15 are in a flat level condition with the opening facing upward from the flat surface).

Viewed another way, such line of weakness or perforation line 25 may be either the same height, slightly above or slightly below the outermost sealed edges 60 of the wrappers 50 (when viewed from such a cross-sectional view as seen in FIG. 1B), while the wrappers are stored in the splittable outer packaging 10 (and also after splitting if so desired). In a desirable embodiment, the sealed edges 60 of wrappers 50 within each of the portions 28, 30 of the splittable outer packaging 10 are aligned with each other within their respective portions, and the sealed edges 60 are also positioned adjacent to the splitting structure (e.g. line of weakness or perforation line 25) of the splittable outer packaging 10. Desirably, sealed edges 60 are parallel with the thickness direction T of the splittable outer packaging 10.

Desirably in one embodiment, the height of the splittable outer packaging 10 before splitting, is between about 140 mm (such as for liners) and 300 mm (such as for incontinence products), alternatively, between about 140 mm and 200 mm (such as for pads). Desirably in one embodiment, the height of each portion 28, 30 is between about 70 mm and 100 mm. In one embodiment, in which the portions are not equal, it is desirable for one portion to be between about 50 mm and 100 mm, while the other to be between about 90 mm and 130 mm.

In one embodiment, the respective heights H1, H2 of the individually-wrapped absorbent articles in multiple adjacent stacks 33, 35, 37 are desirably equal (as in FIGS. 1B, 1D-1). In an alternative embodiment, the heights of the individually-wrapped absorbent articles H1, H2 in stacks may be different (FIG. 1D-2). FIG. 1D-2 also illustrates an embodiment in which the numbers of individually-wrapped absorbent articles in a stack may differ between adjacent stacks, but at least some wrapper sealed edges 60 of adjacent multiple stacks (33, 35) of articles are nevertheless aligned before and after splitting of the splittable outer packaging 48.

The line of weakness or perforation line 25 (splitting structure) may be discretely present or visually apparent to a consumer of the splittable outer packaging 10, 17, 45, 48. It may also serve as a visual dividing line between a first colored package portion 28 and a second, differently colored package portion 30 (as seen for example in the separated outer packages of FIG. 3). It may be used to help a consumer transfigure the relatively large splittable outer packaging 10 into the shorter single outer packaging 11 (of FIG. 5) that is twice as thick as the starting dimensions of the large outer packaging 10, or instead, into the two completely separated shorter outer packages 13, 15 (of FIG. 3) respectively. This assistance is provided by highlighting the two portions that may be separated by the perforation line. Package illustrations which create a sense of motion or the separation functionality may likewise be included on the splittable outer packaging 10 frontside, backside, and other side face panels. Such illustrations may be printed over the splitting structure itself, or adjacent one or both sides of the splitting structure 25. Differences in color between the two package portions 28, 30 may also assist in highlighting to the consumer, the differences in article types contained in each package portion which is to be separated. Alternatively, as seen in FIG. 1E, the line of weakness or perforation line 25 may merely be used to remove a top lid-like portion 70 of the splittable outer packaging 17, from a lower article-containing portion 71 of the splittable outer packaging 17, thereby exposing, at a formed opening, the individually wrapped absorbent articles at exposed, wrapper sealed edges 60.

As seen in FIG. 1B, which shows a cross-sectional view of the splittable outer packaging 10 of FIG. 1A, sealed edges 60 of the wrappers 50 of adjacent stacks desirably in one embodiment, interlap or nest in the splittable outer packaging 10 at a location adjacent the splitting structure 25 (e.g. line of weakness or perforation line 25). Alternatively, in a second embodiment, a majority of sealed edges 60 interlap if more than one stack of absorbent articles is present in the splittable outer packaging 10. In still a further alternative embodiment, occasional sealed edges 60 of adjacent stacks interlap at a location adjacent the line of weakness or perforation line 25. In yet a further embodiment, the sealed wrapper edges 60 of adjacent stacks do not interlap, but instead are in physical contact with sealed edges 60 of adjacent stacks (such as contacting one another or laying against one another) while in the splittable outer packaging 10.

Desirably, in one embodiment, the height H5 of the wrapper body portion 53 is between about 75 mm (such as for liner) and 115 mm (such as for pad). Desirably in one embodiment, the height H4 of the wrapper seal edges 60 is between about 4 mm and 15 mm, alternatively, between about 4 mm and 10 mm. The interlap overlap distance (or height of diagonally adjacent wrapper, sealed edge overlap when viewed from a cross-sectional view) is represented in FIG. 1B by height H3. Desirably, in one embodiment, the interlap overlap distance H3 is between about 3 mm and 7 mm. In an alternative embodiment, the interlap overlap distance is greater than 10 percent of each adjacent sealed edge height H4. A previously noted, in one embodiment, the total height of the wrapper H1, may be larger than the height of the splitting structure H7, such that the height H4 of the sealed edges 60 extend out above the opening edge 27 by a distance H6, following package splitting.

The wrapper thickness (distance) between one outer edge 66 of the wrapper body 53 to the other is represented by T1 in FIG. 1B. Desirably, in one embodiment the wrapper thickness T1 is between about 5 and 20 mm, alternatively, between about 5 and 8 mm. The distance between immediately adjacent and generally parallel wrapper sealed edges 60 (between individually wrapped articles of the same stack) is represented by T2 in FIG. 1B. Desirably, in one embodiment, the distance T2 is between about 5 and 20 mm, alternatively, between about 10 and 15 mm. The distance between the sealed edge 60 of a wrapped individual absorbent article and an outermost wrapper body edge 66 is represented by T3 in FIG. 4B. Desirably, in one embodiment, the distance T3 is between about 2.5 mm and 10 mm, alternatively, between about 2.5 and 8 mm. It should be appreciated that the larger the distances T2, T3, the easier it is for the consumer to grasp an individually-wrapped absorbent article from the outer package. The T2 distances may be larger if in the embodiment, the wrapper sealed edges 60 are exposed for only alternative wrappers at the opening(s). The distances between the interlapping sealed edges 60 of vertically adjacent individually wrapped absorbent articles (from adjacent stacks) may in one embodiment, be the same or less as that of T3. It should be recognized that the above distances may vary by article type. For example, while panty liners may have smaller distance values (as seen in FIG. 1D-2 (at stack 33)), overnight feminine care pads, adult care pads/inserts and pants may have larger distances as a result of the increased bulk of such articles.

In one embodiment, the individually wrapped absorbent articles of the splittable outer packaging 45 are present in a stack of between 8 and 25 articles, for outer packaging containing a single stack 33. For splittable outer packaging 10 containing two stacks 33, 35, it is desirable that the total number of articles contained in the packaging number between 16 and 40. For splittable outer packaging 17 containing three stacks, 33, 35, 37, it is desirable that the total number of articles contained in the packaging number between 60 and 75. For splittable outer packaging containing four stacks (not shown), it is desirable that the total number of articles contained in the packaging number between 40 and 100.

As noted previously, splitting structure alternatives to either a line of weakness or perforation line are contemplated to be within the scope of the inventive splittable outer packaging. For instance, as seen in the side perspective view of FIG. 1F, a splittable outer packaging 10 may include two portions 28, 30 that are pre-formed to include already separated openings (and separated edges). The two portions are brought and held together along their opening edges 25a, 25b via a sealable ribbon or tape 78. In this separated seam embodiment, the ribbon or tape covers and hold the portions 28, 30 together until the splittable outer packaging 10 is desired to be split. In use, the sealed ribbon or tape 78 may be pulled from about the edge of the splittable outer packaging 10 and the upper and lower portions 28, 30 separated and stored where desired.

In a further alternative embodiment as seen in FIG. 1G, the structural feature of the splittable outer packaging 10 that allows for splitting and separation may be a series of lines of weakness or perforation lines 88a, 88b that define a removable strip 79 ending in a pull tab 81. The pull tab may be used to initiate the separation of the removable strip 79 along both perforation lines 88a, 88b around the entire periphery or a portion of the periphery of the splittable outer packaging 10, in a similar manner to the removal of the sealable tape 78 of the previous embodiment.

In yet still a further alternative embodiment as seen in FIG. 1H, the splitting structure may be a perforation line 25 including two separated perforation lines 25c, 25d along at least one package side face panel 16. The separated perforation lines 25c, 25d are placed along the height of the wrapper body portions 53 of the individual wrappers 50 such that upon splitting of the large outer package 10 into a single shorter, or two separate shorter outer packages, the side edges of the wrapper body portions 53 can be clearly seen (as having a height larger than the height of the shorter outer package opening edge defining the package opening). This feature is illustrated in FIG. 1H which shows a splittable outer packaging 10 having separated perforation lines 25c, 25d, surrounding a removable, circular outer package piece 25e (on the side face panel 16). This feature is also illustrated in FIG. 2, in which an alternative embodiment of the splittable outer packaging 10 is shown in a side perspective view. As seen in the figure, the splittable outer packaging 10 has been at least partially separated along three of its perforated edges and a circular end piece of the package has been removed that was adjacent to separated perforation lines 25c and 25d.

Upon splitting of the splittable outer packaging 10, 17 in accordance with the invention, at least one, but in most embodiments two openings (defined by opening edge 27), are formed in the resulting shorter outer packages. The individually wrapped absorbent articles are visible at least from a top view, and in some embodiments as described, from a side view as well. The individually wrapped absorbent articles may be withdrawn, one-wrapped article at-a-time (if desired) from the resulting shorter outer package.

In further alternative embodiments of the splittable outer packaging 10, one or more visual cues may be employed to emphasize the splittable nature of the package and assist the consumer in splitting the package at the appropriate location on the package. Examples of such visual cues are illustrated in FIGS. 1C(1-4) and FIGS. 2, 3, and 5. For instance, visual cues designed to provide emphasis as to the type of motion necessary to open the package include use of directional arrows 70, and zippers 71. Illustrations of zippers 71 may be in an opened format 72, which also optionally show images of individually wrapped articles 74 contained in the splittable outer packaging 10. The zipper teeth 73 may be shown in either a closed or open format. In still a further alternative embodiment, an illustration of a ribbon 76 may be used along the line of perforation 77, so as to emphasize the location of splitting or opening functionality of the outer package. Such printed zippers, ribbons, and arrows are desirably placed on or immediately adjacent to the splitting structure (line of perforation or other separation structure). The visual cues may be used individually or in combination (as seen in FIGS. 2, 3, and 5). Further visual cues may be used to distinguish different portions of the splittable outer packaging 10, such as different package coloration, the absence of color over specific portions, the presence of coatings and finishes (such as for example, different placement of glossy and matte finishes) over specific portions. Packaging portions without color or printed features, may be particularly effective in showing the consumer different article types contained within the splittable outer packaging. Still other additional visual cues may also be utilized on the outer packaging to communicate the storage alternatives available to the consumer following the splitting of the splittable outer packaging (not shown).

In this fashion, a splittable outer packaging is disclosed which may be transfigured to accommodate various consumer, article-storage needs. Such splittable outer packaging may be split to form relatively short trays from which individually-wrapped absorbent articles may be easily retrieved by grasping exposed sealed edges of the individual wrappers enveloping the articles. Since each split package forms a tray having an opening (whether a single outer package or multiple discrete outer packaging) that is approximately the same dimension as the L1 dimension of the individually wrapped article (wrapper and article), removal of the article from the tray is less cumbersome. Such splittable outer packaging does not necessitate specific article orientation within the outer packaging, or complex manufacturing processes. The contemplated splittable outer packaging allows the consumer to maintain relatively fewer numbers of absorbent articles in a clean and organized fashion prior to use, while still taking advantage of a lower cost-per-article, from high article volume purchases.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A splittable outer packaging having a length direction, a height direction, and a thickness direction, and further having at least one side face panel and an upper and lower portion, said splittable outer packaging comprising:
a splitting structure for separating the splittable outer packaging upper and lower portions from one another, said structure included along said at least one side face panel;
at least one stack of individually-wrapped absorbent articles, said absorbent articles each being completely wrapped in a wrapper having at least one wrapper sealed edge, with at least a portion of said individually-wrapped absorbent articles in said at least one stack, having their sealed edges oriented within said splittable outer packaging such as to be parallel with said splittable outer packaging thickness direction and generally adjacent said splitting structure.

2. The splittable outer packaging of claim 1, wherein said splitting structure is selected from the group consisting of a line of weakness, a perforation line, or separated seams held together with a fastening device.

3. The splittable outer packaging of claim 1, wherein said splittable outer packaging includes an upper portion having at least one stack of individually-wrapped absorbent articles, and a lower portion having at least one stack of individually-wrapped absorbent articles.

4. The splittable outer packaging of claim 3, wherein said lower portion has at least two stacks of individually-wrapped absorbent articles.

5. The splittable outer packaging of claim 1, wherein said upper and lower portions each contain different numbers of stacks of individually-wrapped absorbent articles.

6. The splittable outer packaging of claim 1, wherein said upper and lower portions each contain different types of individually-wrapped absorbent articles.

7. The splittable outer packaging of claim 1, wherein the splittable outer packaging includes different packaging appearance for each portion.

8. The splittable outer packaging of claim 1, wherein each portion includes at least one stack of individually-wrapped absorbent articles, and a plurality of wrapper sealed edges of individually-wrapped absorbent articles from at least one stack of each portion, are oriented such that they are parallel with the thickness direction of said splittable outer packaging and generally adjacent said splitting structure.

9. The splittable outer packaging of claim 1, wherein said absorbent articles are folded absorbent articles selected from the group consisting of feminine hygiene absorbent personal care articles and adult incontinence absorbent personal care articles.

10. The splittable outer packaging of claim 1, wherein said absorbent articles are selected from the group consisting of panty liners and sanitary pads.

11. The splittable outer packaging of claim 1, wherein said wrappers include a wrapper body portion, and said wrapper sealed edges extend outwardly away from said wrapper body portion.

12. The splittable outer packaging of claim 1, wherein said wrappers include two opposing wrapper sealed edges.

13. The splittable outer packaging of claim 1, wherein said wrappers include four wrapper sealed edges.

14. The splittable outer packaging of claim 1, wherein said wrappers are visually distinguishable so that adjacent wrappers are visually different from one another.

15. The splittable outer packaging of claim 1, wherein said upper and lower portions each include at least one stack of individually-wrapped absorbent articles, said stacks are generally parallel and adjacent one another, and wrapper sealed edges from each stack at least partially interlap with wrapper sealed edges of an adjacent stack.

16. The splittable outer packaging of claim 1, wherein said splittable outer packaging further includes visual cues to highlight either the presence of, or the functionality of the splitting structure within said splittable outer packaging.

17. The splittable outer packaging of claim 1, wherein said upper and lower portions each include at least one stack of individually-wrapped absorbent articles, and at least the wrapper sealed edges from each stack are colored differently between each portion.

18. The splittable outer packaging of claim 1, wherein said splittable outer packaging includes a splitting structure having a height along said at least one side face panel, and said wrappers include a wrapper height, such that said wrapper height is larger than said splitting structure height.

19. The splittable outer packaging of claim 18, wherein said splitting structure height is uniform about the entire periphery of the splittable outer packaging.

20. The splittable outer packaging of claim 18, wherein said splitting structure height is non-uniform about the entire periphery of the splittable outer packaging.
